(19) United States
(12) Patent Application Publication
(10) Pub. No.: US 2025/0249441 A1
(43) Pub. Date: Aug. 7, 2025

(54) SOLUBLE GERMANIUM CHELATE CATALYST AND PREPARATION METHOD THEREFOR AND USE THEREOF

(71) Applicants: JIANGSU GUOWANG HIGH-TECHNIQUE FIBER CO., LTD., Suzhou (CN); GUOWANG HIGH-TECH FIBER (SUQIAN) CO., LTD., Suqian (CN)

(72) Inventors: Junsong Tang, Suzhou (CN); Shuchang Bian, Suzhou (CN)

(73) Assignees: JIANGSU GUOWANG HIGH-TECHNIQUE FIBER CO., LTD., Suzhou (CN); GUOWANG HIGH-TECH FIBER (SUQIAN) CO., LTD., Suqian (CN)

(21) Appl. No.: 19/018,397

(22) Filed: Jan. 13, 2025

(30) Foreign Application Priority Data
Feb. 6, 2024 (CN) 202410166670.4

(51) Int. Cl. B01J 31/12; B01J 35/27; B01J 35/30; C08G 63/183

(52) U.S. Cl. CPC B01J 31/12; B01J 35/27; B01J 35/394; C08G 63/183

(57) ABSTRACT

A soluble germanium chelate catalyst and a preparation method therefor and use thereof. The preparation method comprises steps of: reacting germanium dioxide with an aqueous solution of an acid to generate a clear solution containing a germanium chelate, wherein the clear solution comprises excess acid; adding a Lewis base to the clear solution to neutralize the excess acid to obtain an aqueous solution of the germanium chelate; post-treating to give the soluble germanium chelate catalyst; the acid is selected from the group consisting of diacids, and C3-C6 diacids or triacids containing hydroxyl or amino group, and combinations thereof. The post-treatment comprises two methods: directly adding ethylene glycol, or cooling and crystallizing the aqueous solution, washing and drying, and then dissolving again in water and ethylene glycol. This catalyst can catalyze the polymerization of PETG or PCTG and significantly improve the viscosity and various hue qualities of the product.

### CROSS-REFERENCE TO RELATED APPLICATIONS

This application claims the benefit of priority to Chinese Patent Application No. 202410166670.4 filed on Feb. 6, 2024, and the entire disclosure of it is hereby incorporated by reference.

### TECHNICAL FIELD

The present disclosure relates to a soluble germanium chelate catalyst and a preparation method therefor and use thereof.

### BACKGROUND

PETG and PCTG are both transparent, amorphous copolyesters. Both the PETG and PCTG can be prepared by esterification and polymerization reactions using terephthalic acid PTA, ethylene glycol EG, and 1, 4-cyclohexanedimethanol CHDM in the presence of esterification and polymerization catalysts. The PETG and PCTG can be distinguished according to the different mole proportions of segments corresponding to CHDM in the total diol (CHDM and EG) segments in the copolyesters. Generally, the polymerization products having segments corresponding to CHDM in a molar ratio of 31% to 32% are called PETG copolyesters; while the polymerization products having segments corresponding to CHDM in a molar ratio of 60% to 62% are called PCTG copolyesters. Both the two polymerization products have high transparency, good toughness and impact strength, excellent low-temperature toughness, high tear resistance, good processing performance, and excellent chemical resistance. Both can be processed using traditional molding methods such as extrusion, injection molding, blow molding, and vacuum forming. Both can be widely used in the markets of plates and sheets, high-performance shrink films, bottles, and profiles; they can also be used to produce toys, household utensils, and medical supplies, etc.

Catalysts used for catalyzing esterification and polymerization reactions for PETG and PCTG typically comprise, for example, antimony-based catalysts, titanium-based catalysts, germanium-based catalysts, tin-based catalysts, and etc. The brightness and non-crystalline properties of polymer products are crucial in the catalytic synthesis of PETG and PCTG.

Antimony-based catalysts are the cheapest and have good catalytic polymerization activity, however, during high-temperature reactions, they are prone to undergo reduction reactions to generate antimony ash, which can result in a low hue L value (generally ≥56.0) and a high b value (generally ≤4.0) of PETG and PCTG polymer products, leading to poor external color and inability to improve the L value of the products through toners, therefore, antimony-based catalysts are usually not used alone during polymerization.

Titanium-based catalysts have high activity, but they also catalyze high levels of side reactions, resulting in more by-products in the products, with yellowish color and a high b value, even if toners are used to adjust, the hue of the products is poor, therefore, titanium-based catalysts are usually not used alone during polymerization.

Tin-based catalysts also suffer from poor fastness to sunlight and yellowing of chips, and are toxic, causing significant environmental pollution.

Germanium-based catalysts are the most commonly used catalysts for synthesizing PETG and PCTG, usually used alone or in combination with other catalysts. Their catalytic effect is relatively mild, the level of side reactions is low, and the hue of the polymer products is relatively good, usually with an L value of ≤63.5.

Germanium dioxide is the most commonly used germanium-based catalyst, and it has the aforementioned advantages, however, it has high hardness and is usually difficult to pulverize the germanium dioxide powder to a particle size below 0.1˜0.3 μm of requirement for solid-phase catalysts. The particle size of commercially available germanium dioxide powder is usually at the level of ≥75 μm, which is too large, and adding the germanium dioxide powder during the later stage of esterification of the self polymerization system will result in poor catalytic effect and prevent the polymerization reaction from being completed.

The current method of using germanium dioxide is adding germanium dioxide powder to distilled water under agitating conditions and refluxing for a long time, so that germanium dioxide reacts with water to form germanic acid (H₂GeO₃ or Ge(OH)₄), with a general concentration of only about 1.0%. And there are three types of germanium dioxide crystals:hexagonal crystal form, tetragonal crystal form and amorphous form, where only tetragonal crystal form has slightly soluble properties in water, and the other two are insoluble. In addition, the specific gravity of the hexagonal crystal form is 4.228, and the specific gravity of the tetragonal crystal form is 6.239, even if the germanium acid is added to the polymerization system in the aforementioned form, it is easy to dehydrate and recover to the solid state of germanium dioxide under high temperature conditions during the esterification and polymerization stages, and after a long time, it will deposit on the reactor wall and the melt pipeline, affecting the long-term operation of the device.

Chinese patent CN104640905A discloses the use of titanium and germanium compounds as composite catalysts for catalyzing the polymerization of PCTG, although it can improve the hue of the product, the hhue performance of the product still needs to be improved.

Chinese patent CN113429549B discloses a composite catalyst for catalyzing the polymerization of poly(cyclohexylene dimethylene terephthalate), the catalyst is a metal/non-metal composite, and is a composite of a first catalyst Zr—Te—P, a second catalyst Si—Zr—B, or a third catalyst Si—Zr—Sn—Ba, although the composite catalyst can improve the polymerization reaction rate and reduce the occurrence of side reactions, the hue performance of the product still needs to be improved.

Chinese patent CN116693830B discloses a quaternary ammonium salt titanium/zirconium chelate catalyst prepared by reacting 2, 3-dihydroxypropyl-trimethylammonium chloride with tetrabutyl titanate or tetrabutyl zirconate, which is used for catalyzing the copolymerization of modified PETG. Although the color of the copolyester product is good, the structure of the catalyst is complex and its preparation process is complicated.

Chinese patent CN1121727A discloses a composite catalyst of manganese (II) acetate, zinc acetate, titanium isopropoxide and germanium dioxide, however, the viscosity of PETG prepared by this catalyst is not high enough.

### SUMMARY OF THE INVENTION

A purpose of the present disclosure is to provide a method for preparing a soluble germanium chelate catalyst, when this catalyst obtained by the preparation method is used to catalyze the polymerization of PETG or PCTG, it can significantly improve the viscosity and various hue qualities of the product, and the catalyst is soluble and will not deposit on the reactor wall or the melt pipeline, which does not affect the long-term operation of the device.

To achieve the above purpose, a technical solution employed by the present disclosure is:
a method for preparing a soluble germanium chelate catalyst, comprises steps of: reacting germanium dioxide with an aqueous solution of an acid to generate a clear solution containing a germanium chelate, wherein the clear solution comprises excess acid; adding a Lewis base to the clear solution to neutralize the excess acid to obtain an aqueous solution of the germanium chelate; post-treating the aqueous solution of the germanium chelate to give the soluble germanium chelate catalyst; the acid is selected from the group consisting of diacids, and C3-C6 diacids or triacids containing hydroxyl or amino group, and combinations thereof.

In the present disclosure, when preparing the above-mentioned aqueous solution of an acid, the dissolution rate can be accelerated by heating.

In some implementations, the acid is selected from the group consisting of citric acid, tartaric acid, glutamic acid, gluconic acid, L-lactic acid, oxalic acid, and combinations thereof.

In some implementations, when the acid is selected from combinations of various acids, it may contain oxalic acid. In the mixed acid, the molar ratio of other acids to oxalic acid is 1:(1-3).

In the prior art, the germanium dioxide powder is difficult to dissolve in water and cannot be prepared into a clear aqueous solution. When used to catalyze the polymerization reaction, germanium dioxide is usually reacted with water to form germanic acid through reflux, however, the concentration of this germanic acid is low, and when added to the polymerization system in the form of germanic acid, it is also prone to dehydrate and recover to the solid state of germanium dioxide under high temperature conditions in the esterification and polymerization stages, which leads to the deposition of germanium dioxide on the reactor wall and the melt pipeline, which, on the one hand, is not conducive to the long-term operation of the device, on the other hand, it also affects the catalytic effect on polymerization, and is not conducive to the hue of polyester products.

The present disclosure chelates germanium dioxide with an aqueous solution of a specific type of acids. The acid in the present disclosure is selected from of diacids, especially oxalic acid, and C3-C6 diacids or triacids containing hydroxyl or amino groups, and combinations thereof, where oxalic acid does not contain hydrophobic alkyl segments, while other diacids or triacids have hydrophilic hydroxyl or amino groups and shorter carbon chains with strong hydrophilicity. Two carboxyl groups in these acids can undergo chelation reactions with germanium atoms in germanium dioxide, generating corresponding germanium chelate, and the hydroxyl or amino groups in the acid can increase the hydrophilicity of the germanium chelate, allowing the reaction generated germanium chelate to dissolve in water, resulting in a clear aqueous solution of the germanium chelate, which can be used to prepare the soluble germanium chelate catalyst of the present disclosure. If the acid has a long hydrophobic alkyl segment, its hydrophilicity may be insufficient, which may cause the germanium chelate to not be completely dissolved in water.

When germanium dioxide reacts with an acid, it is difficult to form a clear solution, and excess acid needs to be added during the reaction, however, excessive acid can affect the polymerization effect of the catalyst when it is used for polymerization, especially for the polymerization of PETG/PCTG. In order to remove excess acid, it can be purified by crystallization, but the process is complicated. This disclose neutralizes the excess acid by adding a Lewis base to the clear solution, which is beneficial for simplifying the preparation process and does not affect the polymerization effect of the obtained chelate catalyst during polymerization. That is, the addition of the Lewis base to neutralize the excess acid enables the preparation of the catalyst without the need for separate crystallization and purification, resulting in the catalyst with sufficient catalytic activity, which simplifies the polymerization process while ensuring the same quality of polymer product.

When the acid is citric acid, the reaction equation is as follows:

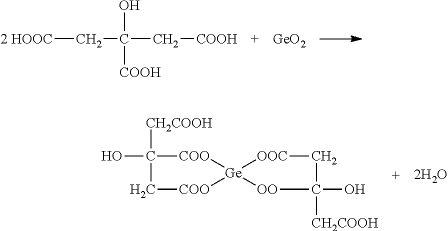

When the acid is tartaric acid, the reaction equation is as follows:

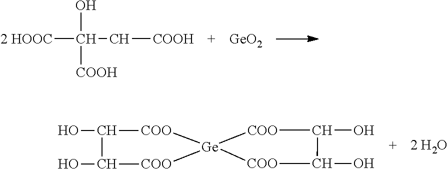

When the acid is oxalic acid, the reaction equation is as follows:

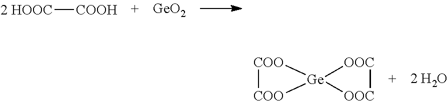

In some implementations, the structure of germanium dioxide is selected from hexagonal crystal form, anatase form or amorphous form.

In some implementations, the particle size of germanium dioxide ranges from 0.1˜75 μm; preferably 0.1˜50 μm, more preferably 0.1˜5 μm.

In some implementations, the molar ratio of germanium dioxide to the acid is 1:(1.0˜5.0); preferably 1:(1.8˜3.5), more preferably 1:(2.2˜2.6).

In some implementations, the reaction is carried out at 30˜100° C.

In some implementations, the reaction is carried out for 2˜6 hours.

In some implementations, the reaction is carried out in a reactor provided with a reflow cooling system, and the water vapor generated by the reaction is refluxed to the reactor; as the reaction proceeds, germanium dioxide gradually reacts to form the germanium chelate and dissolves in water, and the reaction system gradually changes from a white suspension to a clear and transparent state. That is, a stable aqueous solution of the germanium chelate is formed, which will not precipitate when stored at room temperature. The aqueous solution is a stable homogeneous system.

In some implementations, in the aqueous solution of the germanium chelate, the mass concentration of the germanium chelate is 0.5˜12.0%. According to different acid chelating agents, the solubility of the obtained germanium chelate in the aqueous solution may vary, preferably be 1.0˜10.0%, most preferably be 2.0˜8.0%.

In some implementations, the post-treatment comprises a step of adding ethylene glycol to the aqueous solution of the germanium chelate to obtain a germanium chelate solution in a mixed solution of water and ethylene glycol. The liquid catalyst solution obtained by adding ethylene glycol EG to the aqueous solution can be directly used for catalyzing esterification and polymerization reactions for PETG/PCTG. This is the first form of the catalyst in this application, which is a liquid form.

In some implementations, after neutralization with a Lewis base, the pH value of the clear solution is 5.5˜7.0, preferably 5.8˜6.8, and more preferably 6.2˜6.5.

In some implementations, the Lewis base is selected from the group consisting of sodium hydroxide, lithium hydroxide, potassium hydroxide, sodium acetate, calcium acetate, zinc acetate, lithium acetate, manganese acetate, cobalt acetate, sodium carbonate, sodium bicarbonate, magnesium hydroxide, aluminum hydroxide, and combinations thereof.

In some implementations, the post-treatment comprises steps of cooling, standing for crystallization, filtering, washing, and drying the aqueous solution of the germanium chelate. The catalyst obtained by the second post-treatment method is in a crystalline state. When the catalyst in this crystalline state is subsequently used to catalyze the polymerization, it still needs to be dissolved in a mixed solvent of water and ethylene glycol, but the catalyst in crystalline state is convenient for storage and transportation. The purified soluble chelated germanium salt can further remove excess acid in the reaction process, and eliminate the effects of increased esterification and polymerization side reactions caused by residual acid. Furthermore, it can significantly improve the quality of copolyester products and significantly enhance their hue.

In some implementations, the temperature after cooling is −20˜0° C.

In some implementations, the washing is carried out in anhydrous alcohol at 50˜70° C., and excess acid is further removed through washing.

In some implementations, the washing is carried out for 2˜4 hours.

In some implementations, when washing, the mass ratio of the crystal to anhydrous alcohol is 1:(1˜2).

In some implementations, the post-treatment further comprises steps of dissolving the germanium chelate crystal in a certain amount of water under agitating conditions at 60˜80° C., adding ethylene glycol after complete dissolution, and continuing to agitate evenly to obtain the catalyst.

In some implementations, in the soluble germanium chelate catalyst, the mass ratio of water to ethylene glycol is 1:4˜4:1, preferably 3.5:6.5˜6.5:3.5, and more preferably 4.5:5.5˜5.5:4.5.

In some implementations, the addition of ethylene glycol is preferably carried out under agitation. Adding ethylene glycol can reduce the water content in the catalyst solution, ensuring that the catalyst can be directly injected into the polymerization reaction system in the later stage of esterification reaction.

In some implementations, in the catalyst solution prepared by redissolving the crystal in water and ethylene glycol, the mass concentration of the germanium chelate is 0.5%˜14.0%, preferably 1.0%˜10.0%, and more preferably 2.0%˜8.0%. The catalyst solution is added with EG again before use, and the final content of soluble chelated germanium salt in the mixed solvent of water and EG is controlled to be 1.0˜3.0%, under this concentration condition, it is convenient to inject with industrial catalyst devices.

The present disclosure further provides a soluble germanium chelate catalyst prepared by the method for preparing a soluble germanium chelate catalyst mentioned above. When this catalyst is used to catalyze the polymerization of PETG or PCTG, it can significantly improve the viscosity and various hue qualities of the product, and the catalyst is soluble and will not deposit on the reactor wall or the melt pipeline, which does not affect the long-term operation of the device.

The present disclosure further provides a method for synthesizing a PETG or PCTG polyester, the preparation method comprises steps of: reacting germanium dioxide with an aqueous solution of an acid to generate a clear solution containing a germanium chelate, wherein the clear solution comprises excess acid; adding a Lewis base to the clear solution to neutralize the excess acid to obtain an aqueous solution of the germanium chelate; post-treating the aqueous solution of the germanium chelate to give a soluble germanium chelate catalyst; the acid is selected from the group consisting of diacids, and C3-C6 diacids or triacids containing hydroxyl or amino group, and combinations thereof; using terephthalic acid, ethylene glycol, and 1,4-cyclohexanedimethanol as polymerization monomers, conducting esterification and polymerization reactions in the presence of a catalyst to obtain the PETG or PCTG, where the catalyst comprises the soluble germanium chelate catalyst.

In some implementations, the soluble germanium chelate catalyst is used alone or in combination with a titanium-based catalyst.

In the present disclosure, PETG refers to a copolyester of terephthalic acid PTA, ethylene glycol EG, and 1, 4-cyclohexanedimethanol CHDM in which the segments corresponding to CHDM account for 31% to 32% by mole of the total diol (CHDM and EG) segments; PCTG refers to a copolyester of terephthalic acid PTA, ethylene glycol EG, and 1, 4-cyclohexanedimethanol CHDM in which the segments corresponding to CHDM account for 60% to 62% by mole of the total diol (CHDM and EG) segments.

When the catalyst of the present disclosure is used for the above purposes, it has better catalytic activity and reaction efficiency, the catalyst maintains a stable homogeneous state during esterification and polymerization processes, and can catalyze polymerization reactions more efficiently, and compared with solid-phase germanium dioxide, the efficiency is improved by 15˜20%.

In some implementations, the soluble germanium chelate catalyst is used alone as the esterification catalyst and polymerization catalyst, and in terms of germanium dioxide, the esterification catalyst accounts for 5˜15 ppm of the polymer mass, and the polymerization catalyst accounts for 50˜70 ppm of the polymer mass.

In some implementations, the soluble germanium chelate catalyst is used in combination with a titanium-based catalyst, and the titanium element in the titanium-based catalyst and the germanium element in the soluble germanium chelate catalyst account for 1˜35 ppm and 150˜10 ppm of the polymer mass, respectively, preferably 3˜20 ppm and 100˜25 ppm, respectively; more preferably 5˜15 ppm and 50˜35 ppm, respectively.

Due to the use of the above technical solutions, the present disclosure has the following advantages over the prior art:

The present disclosure involves a chelation reaction between germanium dioxide and an aqueous solution of a specific type of acid, the acid is selected from hydrophilic diacids or triacids with chelating properties, and two carboxyl groups in these acids can undergo chelation reactions with germanium atoms in germanium dioxide, generating corresponding germanium chelate, and the hydroxyl or amino groups in the acid can increase the hydrophilicity of the germanium chelate, allowing the reaction generated germanium chelate to dissolve in water, resulting in a clear aqueous solution of the germanium chelate, so that the soluble germanium chelate catalyst of the present disclosure is in a form of a dissolved germanium chelate solution. When used to catalyze the polyester synthesis, it will not deposit solids on the reactor wall or the melt pipeline, and will not affect the long-term operation of the device.

The soluble germanium chelate catalyst of the present disclosure has high stability and does not produce precipitation even after being stored at room temperature for 2 months. When the acid is citric acid, the corresponding catalyst does not produce precipitation even after being stored at 100-280° C. for one month, and has excellent stability and heat resistance.

The soluble germanium chelate catalyst of the present disclosure can maintain a homogeneous solution state throughout the esterification and polymerization processes, and its catalytic efficiency is superior to traditional germanium-based catalysts such as germanium dioxide, the catalytic activity can be increased by 15%˜20%, and the amount of germanium-based catalyst can be effectively reduced while ensuring the same catalytic efficiency, which can significantly reduce the cost of the catalyst on account of that germanium-based catalysts are expensive.

When the acid is used in a chemical excess ratio, removing the excess acid during the preparation of the catalyst in the present disclosure can avoid the adverse effects of excess acid on the hue of polyester product.

When the soluble germanium chelate catalyst of the present disclosure is used for catalyzing the copolymerization synthesis of PETG or PCTG, a polyester product with high viscosity and good hue quality can be obtained.

By using the soluble germanium chelate catalyst of this application, higher concentration aqueous solutions can be prepared, when used for the polymerization of PETG or PCTG, compared with traditional germanium-based catalysts, better or similar polymerization effects can be achieved with lower catalyst dosage.

### DETAILED DESCRIPTION OF EXEMPLARY EMBODIMENTS

The present disclosure is further explained in detail below in combination with specific embodiments; it should be understood that, those embodiments are to explain the basic principle, major features and advantages of the present disclosure, and the present disclosure is not limited by the scope of the following embodiments; the implementation conditions employed by the embodiments may be further adjusted according to particular requirements, and undefined implementation conditions usually are conditions in conventional experiments. In the following embodiments, unless otherwise specified, all raw materials are basically commercially available or prepared by conventional methods in the field.

The embodiments described below are only for illustrating the technical concepts and features of the present disclosure, and are intended to make a person familiar with the technology being able to understand the content of the present disclosure and thereby implement it, and should not limit the protective scope of this disclosure. Any equivalent variations or modifications according to the spirit of the present disclosure should be covered by the protective scope of the present disclosure.

### Embodiment 1

This embodiment provided a soluble germanium chelate catalyst, which was used in the polymerization of PETG or PCTG, and was prepared by specific steps as follows:

### Preparation of the Catalyst:

A certain amount of distilled water was injected into a reactor provided with a reflow cooling system, stirring was carried out in the reactor and the temperature was raised to 70° C., and the weighed tartaric acid was added into the reactor and agitated to dissolve for 2.0 hours. Then the weighed germanium dioxide powder was slowly added into the reactor, and the water vapor generated during the reaction process was cooled by reflux and returned to the reactor. During the reaction process, germanium dioxide gradually reacted to form a germanium chelate, which was dissolved in water, after 2.0 hours of reaction, the white suspension gradually became clear and transparent, forming a stable tartaric acid chelated germanium salt solution, and the aqueous solution was left at room temperature for 2 months without precipitation. The mass concentration of the tartaric acid chelated germanium salt in the aqueous solution was controlled to 13.08%. The pH of the chelated germanium salt solution was adjusted to 6.5 using sodium acetate. Under agitating conditions, the temperature of the chelated germanium salt solution was maintained at 80° C., and ethylene glycol EG was metered and injected into it to obtain a tartaric acid chelated germanium salt catalyst solution, in which the mass concentration of tartaric acid chelated germanium salt is 7.05% (the content was about 2.0% in terms of germanium dioxide).

### Petg Polymerization:

Continuous polymerization was carried out on a 30,000 ton/year PETG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1, 4-cyclohexanedimethanol CHDM in a molten state were mixed in a molar ratio of PTA:EG:CHDM=1:0.93:0.32 (where EG was in an excess feed ratio, all CHDM entered the corresponding segments in the copolyester, and the mole percentage of segments corresponding to CHDM in the total diol segments was 32%), and in a total molar ratio of PTA:(EG+CHDM)=1:1.25, the raw materials were accurately metered and slurried, the prepared tartaric acid chelated germanium salt catalyst solution was injected into a slurry convey pipeline as an esterification catalyst, with an amount of 10 ppm (in terms of germanium dioxide) of the total mass of the melt, the prepared slurry was conveyed to the first esterification reactor through a screw metering pump, with the temperature in the first esterification reactor being controlled at 254˜255° C. and the process column pressure being 70˜80 kPa (positive pressure), the second esterification reactor was designed with multi-chambers having three chambers for easy addition of various additives, the prepared tartaric acid chelated germanium salt catalyst solution was added to the second chamber of the second esterification reactor, for the polymerization catalyst (added in the second chamber to facilitate evaporation of water in the catalyst), the amount of polymerization catalyst was 60 ppm (in terms of germanium dioxide) of the total mass of the melt, a toner and a stabilizer (phosphate ester, etc.) were added into the third chamber of the second esterification reactor, the second esterification reactor was a normal-pressure reactor, and the esterification temperatures in the three chambers were controlled as follows: 255˜256° C. in the first chamber, 253˜254° C. in the second chamber, and 257˜258° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 258˜260° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 260˜262° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 265˜268° C., the vacuum degree was controlled to be 90˜110 Pa, the dynamic viscosity of the melt at the outlet was controlled to 635˜650 Pa·s, the intrinsic viscosity of PETG at the outlet was controlled to 0.799 (determined in a mixed solvent of phenol:tetrachloroethylene with a volume ratio of 3:2). The indicators for PETG chip are shown in Table 1 below.

### Or Pctg Polymerization:

Continuous polymerization was carried out on a 30,000 ton/year PCTG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1,4-cyclohexanedimethanol CHDM in a molten state were mixed in a molar ratio of PTA:EG:CHDM=1:0.63:0.63 (where EG was in an excess feed ratio, in the copolyester, the mole percentage of segments corresponding to CHDM in the total diol segments was 62%), and in a total molar ratio of PTA:(EG+CHDM)=1:1.26, the raw materials were accurately metered and slurried, the prepared tartaric acid chelated germanium salt catalyst solution was injected into a slurry convey pipeline as an esterification catalyst, with an amount of 10 ppm (in terms of germanium dioxide) of the total mass of the melt, the prepared slurry was conveyed to the first esterification reactor through a screw metering pump, with the temperature in the first esterification reactor being controlled at 256˜257° C. and the process column pressure being 70˜80 kPa (positive pressure), the second esterification reactor was designed with multi-chambers having three chambers for easy addition of various additives, the prepared tartaric acid chelated germanium salt catalyst solution was added to the second chamber of the second esterification reactor, for the polymerization catalyst (added in the second chamber to facilitate evaporation of water in the catalyst, preventing moisture in the catalyst from being detrimental to polymerization; the solubility of the germanium chelate in pure ethylene glycol is not high, in order to make the germanium chelate soluble, water and ethylene glycol are used as a mixed solvent in the preparation of the catalyst), the amount of polymerization catalyst was 60 ppm (in terms of germanium dioxide) of the total mass of the melt, a toner and a stabilizer (phosphate ester, etc.) were added into the third chamber of the second esterification reactor, the second esterification reactor was a normal-pressure reactor, and the esterification temperatures in the three chambers were controlled as: 255˜256° C. in the first chamber, 253˜254° C. in the second chamber, and 260˜262° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 266˜268° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 272˜274° C., the prepolymer from the reaction in the second prepolymerization reactor were introduced into the high viscosity polymerization reactor through the prepolymerization melt pump and the prepolymerization filter, the high viscosity polymerization reactor adopted a two-shaft horizontal disc reactor with the shafts respectively arranged in front and rear portions, the temperature at the outlet of the high viscosity polymerization reactor was controlled to be 280˜282° C., the vacuum degree was controlled to be 90˜110 Pa, the dynamic viscosity of the melt at the outlet was controlled to 600˜620 Pa·s, the intrinsic viscosity of PCTG at the outlet was controlled to 0.802 (determined in a mixed solvent of phenol:tetrachloroethylene with a volume ratio of 3:2). The indicators for PCTG chip are shown in Table 2 below.

### Embodiment 2

Embodiment 2 provided a soluble germanium chelate catalyst, which was used in the polymerization of PETG or PCTG, and was prepared by specific steps basically the same as in Embodiment 1, by only differing in that: when preparing the catalyst, tartaric acid was replaced with citric acid, and the mass concentration of the citric acid chelated germanium salt in the aqueous solution was controlled to 16.06%. After adding EG, the mass concentration of the citric acid chelated germanium salt in the final catalyst solution was 8.66% (the content was about 2.0% in terms of germanium dioxide). Wherein, the citric acid chelated germanium salt aqueous solution can be stored for 3 months at 280° C. without settling. It has excellent stability and heat resistance. It was used to polymerize PETG or PCTG, and the indicators for their chips are shown in Tables 1-2 below.

### Embodiment 3

Embodiment 3 provided a soluble germanium chelate catalyst, which was used in the polymerization of PETG or PCTG, and was prepared by specific steps basically the same as in Embodiment 1, by only differing in that: when preparing the catalyst, tartaric acid was replaced with glutamic acid, and the mass concentration of the glutamic acid chelated germanium salt in the aqueous solution was controlled to 12.87%. After adding EG, the mass concentration of the glutamic acid chelated germanium salt in the final catalyst solution was 6.94% (the content was about 2.0% in terms of germanium dioxide). Wherein, the glutamic acid chelated germanium salt aqueous solution can be stored for 1 month at room temperature without settling. It was used to polymerize PETG or PCTG, and the indicators for their chips are shown in Tables 1-2 below.

### Embodiment 4

Embodiment 4 provided a soluble germanium chelate catalyst, which was used in the polymerization of PETG or PCTG, and was prepared by specific steps basically the same as in Embodiment 1, by only differing in that: when preparing the catalyst, tartaric acid was replaced with oxalic acid, and the mass concentration of the oxalic acid chelated germanium salt in the aqueous solution was controlled to 8.82%. After adding EG, the mass concentration of the oxalic acid chelated germanium salt in the final catalyst solution was 4.76% (the content was about 2.0% in terms of germanium dioxide). Wherein, the oxalic acid chelated germanium salt aqueous solution can be stored for 3 months at room temperature without settling. It was used to polymerize PETG or PCTG, and the indicators for their chips are shown in Tables 1-2 below.

### Embodiment 5

Embodiment 5 provided a soluble germanium chelate catalyst, which was used in the polymerization of PETG or PCTG, and was prepared by specific steps basically the same as in Embodiment 1, by only differing in that: when preparing the catalyst, tartaric acid was replaced with a mixed acid of tartaric acid and oxalic acid, wherein the molar ratio of tartaric acid to oxalic acid was 1:2. The mass concentration of the mixed acid chelated germanium salt in the aqueous solution was controlled to 10.24%. After adding EG, the mass concentration of the mixed acid chelated germanium salt in the final catalyst solution was 5.52% (the content was about 2.0% in terms of germanium dioxide). Wherein, the mixed acid chelated germanium salt aqueous solution can be stored for 3 months at room temperature without settling. It was used to polymerize PETG or PCTG, and the indicators for their chips are shown in Tables 1-2 below.

### Embodiment 6

Embodiment 6 provided a soluble germanium chelate catalyst, which was used in the polymerization of PETG or PCTG, and was prepared by specific steps basically the same as in Embodiment 1, by only differing in that: when preparing the catalyst, tartaric acid was replaced with a mixed acid of citric acid and oxalic acid, wherein the molar ratio of citric acid to oxalic acid was 1:3. The mass concentration of the mixed acid chelated germanium salt in the aqueous solution was controlled to 10.63%. After adding EG, the mass concentration of the mixed acid chelated germanium salt in the final catalyst solution was 5.73% (the content was about 2.0% in terms of germanium dioxide). Wherein, the mixed acid chelated germanium salt aqueous solution can be stored for 3 months at room temperature without settling. It was used to polymerize PETG or PCTG, and the indicators for their chips are shown in Tables 1-2 below.

### Embodiment 7

This embodiment combined the soluble germanium chelate catalyst of Embodiment 1 with a traditional titanium-based catalyst for the polymerization of PETG or PCTG, and the specific steps were as follows:

### Petg Polymerization:

Continuous polymerization was carried out on a 30,000 ton/year PETG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1, 4-cyclohexanedimethanol CHDM in a molten state were mixed in a molar ratio of PTA:EG:CHDM=1:0.93:0.32, and in a total molar ratio of PTA:(EG+CHDM)=1:1.25, the raw materials were accurately metered and slurried, a titanium-based catalyst tetrabutyl titanate was injected into a slurry convey pipeline as an esterification catalyst, with an amount of 3 ppm (in terms of titanium element) of the total mass of the melt, the prepared slurry was conveyed to the first esterification reactor through a screw metering pump, with the temperature in the first esterification reactor being controlled at 254˜255° C. and the process column pressure being 70˜80 kPa (positive pressure), the second esterification reactor was designed with multi-chambers having three chambers for easy addition of various additives, the tartaric acid chelated germanium salt catalyst solution in Embodiment 1 was added to the second chamber of the second esterification reactor (added in the second chamber to facilitate evaporation of water in the catalyst) as part of the polymerization catalyst, and accounted for 40 ppm (in terms of germanium dioxide) of the total mass of the melt, a toner and a stabilizer (phosphate ester, etc.) were added into the third chamber of the second esterification reactor, and at the same time, the titanium-based catalyst tetrabutyl titanate was added to the third chamber of the second esterification reactor as part of the polymerization catalyst, and accounted for 12 ppm (in terms of titanium element) of the total mass of the melt. The second esterification reactor was a normal-pressure reactor, and the esterification temperatures in the three chambers were controlled as follows: 255˜256° C. in the first chamber, 253˜254° C. in the second chamber, and 257˜258° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 258˜260° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 260˜262° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 265˜268° C., the vacuum degree was controlled to be 90˜110 Pa, the dynamic viscosity of the melt at the outlet was controlled to 635˜650 Pa·s, the intrinsic viscosity of PETG at the outlet was controlled to 0.783 (determined in a mixed solvent of phenol:tetrachloroethylene with a volume ratio of 3:2). The indicators for PETG chip are shown in Table 1 below.

### Or Pctg Polymerization:

Continuous polymerization was carried out on a 30,000 ton/year PCTG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1,4-cyclohexanedimethanol CHDM in a molten state were mixed in a molar ratio of PTA:EG:CHDM=1:0.63:0.63, and in a total molar ratio of PTA:(EG+CHDM)=1:1.26, the raw materials were accurately metered and slurried, a titanium-based catalyst tetrabutyl titanate was injected into a slurry convey pipeline as an esterification catalyst, with an amount of 3 ppm (in terms of titanium element) of the total mass of the melt, the prepared slurry was conveyed to the first esterification reactor through a screw metering pump, with the temperature in the first esterification reactor being controlled at 256˜257° C. and the process column pressure being 70˜80 kPa (positive pressure), the second esterification reactor was designed with multi-chambers having three chambers for easy addition of various additives, the tartaric acid chelated germanium salt catalyst solution in Embodiment 1 was added to the second chamber of the second esterification reactor (added in the second chamber to facilitate evaporation of water in the catalyst) as part of the polymerization catalyst, and accounted for 40 ppm (in terms of germanium dioxide) of the total mass of the melt, a toner and a stabilizer (phosphate ester, etc.) were added into the third chamber of the second esterification reactor, and at the same time, the titanium-based catalyst tetrabutyl titanate was added to the third chamber of the second esterification reactor as part of the polymerization catalyst, and accounted for 12 ppm (in terms of titanium element) of the total mass of the melt. The second esterification reactor was a normal-pressure reactor, and the esterification temperatures in the three chambers were controlled as follows: 255˜256° C. in the first chamber, 253˜254° C. in the second chamber, and 260˜262° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 266˜268° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 272˜274° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 280˜282° C., the vacuum degree was controlled to be 90˜110 Pa, the dynamic viscosity of the melt at the outlet was controlled to 600˜620 Pa·s, the intrinsic viscosity of PCTG at the outlet was controlled to 0.802 (determined in a mixed solvent of phenol:tetrachloroethylene with a volume ratio of 3:2). The indicators for PCTG chip are shown in Table 1 below.

### Embodiment 8

Embodiment 8 provided a soluble germanium chelate catalyst, which was used in the polymerization of PETG or PCTG, and was prepared by specific steps basically the same as in Embodiment 1, by only differing in that: when preparing the catalyst, sodium acetate was not used to adjust the pH, and excess acid was removed by concentration, crystallization, and hot ethanol washing. The specific steps for preparing the catalyst were as follows:

A certain amount of distilled water was injected into a reactor provided with a reflow cooling system, stirring was carried out in the reactor and the temperature was raised to 70° C., and the weighed tartaric acid was added into the reactor and agitated to dissolve for 2.0 hours. Then the weighed germanium dioxide powder was slowly added into the reactor, and the water vapor generated during the reaction process was cooled by reflux and returned to the reactor. During the reaction process, germanium dioxide gradually reacted to form a germanium chelate, which was dissolved in water, after 2.0 hours of reaction, the white suspension gradually became clear and transparent, forming a stable tartaric acid chelated germanium salt solution, and the aqueous solution was left at room temperature for 2 months without precipitation. The mass concentration of the tartaric acid chelated germanium salt in the aqueous solution was controlled to 13.08%.

The aqueous solution of tartaric acid chelated germanium salt was concentrated, slowly cooled to −20° C., stood to crystallize, and was filtered, and the crystal was collected, vacuum dried, and then crushed. The crushed crystal was dissolved in anhydrous ethanol at 70° C. under agitating conditions, with a mass ratio of 1:2.0 between the crystal and anhydrous ethanol, the system was agitated for 4.0 hours, and then filtered, excess tartaric acid in the raw material was dissolved in anhydrous ethanol, and the resulting filtrate was cooled and recrystallized again. The steps of dissolution, cooling, and recrystallization for the undissolved crystal solid obtained by filtration were repeated. The crystals obtained twice were combined, vacuum dried and crushed to obtain a soluble tartaric acid chelated germanium salt crystal.

Under agitating conditions, the aforementioned tartaric acid chelated germanium salt crystal was dissolved in hot distilled water at 80° C., with a mass ratio of 1:6.59 between the crystal and distilled water. After the crystal was completely dissolved in distilled water for 1 hour, under agitating conditions, ethylene glycol EG was added into the aqueous solution and agitated for 2 hours, to obtain a tartaric acid chelated germanium salt catalyst solution, in which the mass concentration of tartaric acid chelated germanium salt is 7.05% (the content was about 2.0% in terms of germanium dioxide).

When it was used to polymerize PETG or PCTG, the results are shown in Tables 1-2 below.

### Embodiments 9-13

Embodiments 9-13 provided soluble germanium chelate catalysts, which were used in the polymerization of PETG or PCTG, and were prepared by specific steps basically the same as in Embodiments 2-6, by only differing in that: when preparing the catalyst, sodium acetate was not used to adjust the pH, and excess acid was removed by concentration, crystallization, and recrystallization. The specific operation of removing excess acid by concentration, crystallization, and recrystallization was the same as Embodiment 8.

### Embodiment 14

Embodiment 14 combined the soluble germanium chelate catalyst from Embodiment 8 with a traditional titanium-based catalyst for the polymerization of PETG or PCTG, and the specific steps were the same as Embodiment 7. The results are shown in Tables 1-2 below.

### Comparative Example 1

Comparative Example 1 provided a method for polymerizing PETG or PCTG, which had the same polymerization steps as Embodiment 1, by only differing in that: the esterification and polymerization catalysts were replaced with an aqueous solution of germanium dioxide, and the amount of catalysts used was increased. The aqueous solution of germanium dioxide was prepared by adding germanium dioxide powder to distilled water and conducting a reflux reaction for 8˜10 h, reacting germanium dioxide with water to form germanic acid. The mass concentration of germanium dioxide was 0.8%. The esterification catalyst accounted for 15 ppm (in terms of germanium dioxide) of the total mass of the melt, and polymerization catalyst accounted for 120 ppm (in terms of germanium dioxide) of the total mass of the melt. The polymerization results are shown in Tables 1-2 below.

### Comparative Example 2

Comparative Example 2 provided a method for polymerizing PETG or PCTG, which had the same polymerization steps as Embodiment 1, by only differing in that: the esterification and polymerization catalysts were replaced with a solution of germanium dioxide in a mixed solvent of water and EG, and the amount of catalysts used was increased. The solution of germanium dioxide in a mixed solvent of water and EG was prepared by the following method: adding germanium dioxide powder to distilled water, heating until the germanium dioxide power is completely dissolved, adding EG under agitating conditions, with a mass ratio of water to EG of 1:1, continuing to agitate and dissolve to obtain the solution in the mixed solvent. In the solution, the mass concentration of germanium dioxide was 0.8%. The esterification catalyst accounted for 15 ppm (in terms of germanium dioxide) of the total mass of the melt, and polymerization catalyst accounted for 120 ppm (in terms of germanium dioxide) of the total mass of the melt. The polymerization results are shown in Tables 1-2 below.

### Comparative Example 3

Comparative Example 3 provided a method for polymerizing PETG or PCTG, which had the same polymerization steps as Embodiment 1, by only differing in the types of esterification and polymerization catalysts (a titanium-antimony composite catalyst), timing and dosage of addition, and polymerization process parameters.

### Specifically:

### Petg Polymerization:

Continuous polymerization was carried out on a 30,000 ton/year PETG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1, 4-cyclohexanedimethanol CHDM in a molten state were mixed in a molar ratio of PTA:EG:CHDM=1:0.93:0.32, and in a total molar ratio of PTA:(EG+CHDM)=1:1.25, the raw materials were accurately metered and slurried, a titanium-based catalyst tetrabutyl titanate was injected into a slurry convey pipeline as an esterification catalyst, with an amount of 3 ppm (in terms of titanium element) of the total mass of the melt, the prepared slurry was conveyed to the first esterification reactor through a screw metering pump, with the temperature in the first esterification reactor being controlled at 254˜255° C. and the process column pressure being 70˜80 kPa (positive pressure), the second esterification reactor was designed with multi-chambers having three chambers for easy addition of various additives, ethylene glycol antimony was added to the third chamber of the second esterification reactor as the polymerization catalyst, and accounted for 225 ppm (in terms of antimony element) of the total mass of the melt, a toner and a stabilizer (phosphate ester, etc.) were added into the third chamber of the second esterification reactor, the second esterification reactor was a normal-pressure reactor, and the esterification temperatures in the three chambers were controlled as follows: 255˜256° C. in the first chamber, 253˜254° C. in the second chamber, and 257˜258° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 262˜263° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 264˜265° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 275˜277° C., the vacuum degree was controlled to be 90˜110 Pa, the dynamic viscosity of the melt at the outlet was controlled to 635˜650 Pa·s, the intrinsic viscosity of PETG at the outlet was controlled to 0.780 (determined in a mixed solvent of phenol:tetrachloroethylene with a volume ratio of 3:2). The indicators for PETG chip are shown in Table 1 below.

### Or Pctg Polymerization:

Continuous polymerization was carried out on a 30,000 ton/year PCTG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1, 4-cyclohexanedimethanol CHDM in a molten state were mixed in a molar ratio of PTA:EG:CHDM=1:0.63:0.63, and in a total molar ratio of PTA:(EG+CHDM)=1:1.26, the raw materials were accurately metered and slurried, a titanium-based catalyst tetrabutyl titanate was injected into a slurry convey pipeline as an esterification catalyst, with an amount of 3 ppm (in terms of titanium element) of the total mass of the melt, the prepared slurry was conveyed to the first esterification reactor through a screw metering pump, with the temperature in the first esterification reactor being controlled at 256˜257° C. and the process column pressure being 70˜80 kPa (positive pressure), the second esterification reactor was designed with multi-chambers having three chambers for easy addition of various additives, ethylene glycol antimony was added to the third chamber of the second esterification reactor as the polymerization catalyst, and accounted for 240 ppm (in terms of antimony element) of the total mass of the melt, a toner and a stabilizer (phosphate ester, etc.) were added into the third chamber of the second esterification reactor, the second esterification reactor was a normal-pressure reactor, and the esterification temperatures in the three chambers were controlled as follows: 255˜256° C. in the first chamber, 253˜254° C. in the second chamber, and 260˜262° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 266˜268° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 272˜274° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 280˜282° C., the vacuum degree was controlled to be 90˜110 Pa, the dynamic viscosity of the melt at the outlet was controlled to 600˜620 Pa·s, the intrinsic viscosity of PCTG at the outlet was controlled to 0.806 (determined in a mixed solvent of phenol:tetrachloroethylene with a volume ratio of 3:2). The indicators for PCTG chip are shown in Table 2 below.

### Comparative Example 4

Comparative Example 4 provided a method for polymerizing PETG or PCTG, which had the same polymerization steps as Embodiment 1, by only differing in the types of esterification and polymerization catalysts (a titanium-based catalyst), timing and dosage of addition, and polymerization process parameters. Specifically:

### Petg Polymerization:

Continuous polymerization was carried out on a 30,000 ton/year PETG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1, 4-cyclohexanedimethanol CHDM in a molten state were mixed in a molar ratio of PTA:EG:CHDM=1:0.93:0.32, and in a total molar ratio of PTA:(EG+CHDM)=1:1.25, the raw materials were accurately metered and slurried, a titanium-based catalyst tetrabutyl titanate was injected into a slurry convey pipeline as an esterification catalyst, with an amount of 3 ppm (in terms of titanium element) of the total mass of the melt, the prepared slurry was conveyed to the first esterification reactor through a screw metering pump, with the temperature in the first esterification reactor being controlled at 254˜255° C. and the process column pressure being 70˜80 kPa (positive pressure), the second esterification reactor was designed with multi-chambers having three chambers for easy addition of various additives, the titanium-based catalyst tetrabutyl titanate was added to the third chamber of the second esterification reactor as the polymerization catalyst, and accounted for 20 ppm (in terms of titanium element) of the total mass of the melt, a toner and a stabilizer (phosphate ester, etc.) were added into the third chamber of the second esterification reactor, the second esterification reactor was a normal-pressure reactor, and the esterification temperatures in the three chambers were controlled as follows: 255˜256° C. in the first chamber, 253˜254° C. in the second chamber, and 257˜258° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 260˜261° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 261˜262° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 265˜267° C., the vacuum degree was controlled to be 90˜110 Pa, the dynamic viscosity of the melt at the outlet was controlled to 630˜650 pa·s, the intrinsic viscosity of PETG at the outlet was controlled to 0.780 (determined in a mixed solvent of phenol:tetrachloroethylene with a volume ratio of 3:2). The indicators for PETG chip are shown in Table 1 below.

### Or Pctg Polymerization:

Continuous polymerization was carried out on a 30,000 ton/year PCTG continuous polymerization plant, which comprised a first esterification reactor, a second esterification reactor (a three-chamber structure), a first prepolymerization reactor, a second prepolymerization reactor, and a high viscosity polymerization reactor.

Firstly, pure terephthalic acid PTA, ethylene glycol EG, and 1, 4-cyclohexanedimethanol CHDM in a molten state were mixed in a molar ratio of PTA:EG:CHDM=1:0.63:0.63, and in a total molar ratio of PTA:(EG+CHDM)=1:1.26, the raw materials were accurately metered and slurried, a titanium-based catalyst tetrabutyl titanate was injected into a slurry convey pipeline as an esterification catalyst, with an amount of 5 ppm (in terms of titanium element) of the total mass of the melt, the prepared slurry was conveyed to the first esterification reactor through a screw metering pump, with the temperature in the first esterification reactor being controlled at 256˜257° C. and the process column pressure being 70˜80 kPa (positive pressure), the second esterification reactor was designed with multi-chambers having three chambers for easy addition of various additives, the titanium-based catalyst tetrabutyl titanate was added to the third chamber of the second esterification reactor (added in the third chamber to facilitate evaporation of water in the catalyst) as the polymerization catalyst, and accounted for 20 ppm (in terms of titanium element) of the total mass of the melt, a toner and a stabilizer (phosphate ester, etc.) were added into the third chamber of the second esterification reactor, the second esterification reactor was a normal-pressure reactor, and the esterification temperatures in the three chambers were controlled as follows: 255˜256° C. in the first chamber, 253˜254° C. in the second chamber, and 260˜262° C. in the third chamber; the esterified materials after the esterification reactions were introduced into the first prepolymerization reactor, which was a vertical agitating structure with an inner and outer chamber design, the reaction temperature in the first prepolymerization reactor was controlled to be 266˜268° C. and the vacuum degree was 9.0 kPa, the oligomer polymerized in the first prepolymerization reactor was introduced into the second prepolymerization reactor, which was a single-axis horizontal disc reactor, the reaction temperature in the second prepolymerization reactor was controlled to be 271˜273° C., the prepolymers from the reaction in the second prepolymerization reactor were introduced into the high viscosity polymerization reactor through the prepolymerization melt pump and the prepolymerization filter, the high viscosity polymerization reactor adopted a front-rear two-shaft horizontal disc reactor, the temperature at the outlet of the high viscosity polymerization reactor was controlled to be 277˜278° C., the vacuum degree was controlled to be 90˜110 Pa, the dynamic viscosity of the melt at the outlet was controlled to 610˜630 pa·s, the intrinsic viscosity of PCTG at the outlet was controlled to 0.800 (determined in a mixed solvent of phenol:tetrachloroethylene with a volume ratio of 3:2). The indicators for PCTG chip are shown in Table 2 below.

The polyester melts obtained from the respective embodiments and comparative examples was sliced, and properties of chips were tested using GB/T 14190-2017 standard, and the results are shown in Tables 1-2 below, where IV refers to intrinsic viscosity measured in a mixed solvent of phenol and tetrachloroethylene with a volume ratio of 3:2, and DEG, H₂O, ash content, Fe, and agglomerated particles refer to the mass fraction content of diethylene glycol, water, ash, Fe element, and agglomerated particles in the polyester, respectively.

TABLE 1
Physical and chemical indicators of PETG

| | | | | | | | Agglomerated | | | |
|---|---|---|---|---|---|---|---|---|---|---|
| | IV/ | DEG/ | End carboxyl | H₂O/ | Ash/ | Fe/ | particles ≤10 | Hue | | |
| | dL/g | % | content/mol/t | % | % | mg/kg | μm/% | L | a | b |
| Embodiment 1 | 0.779 | 1.225 | 14.96 | 0.63 | 0.01 | 1 | 0.0 | 65.07 | −1.69 | −0.70 |
| Embodiment 2 | 0.782 | 1.224 | 14.57 | 0.77 | 0.02 | 1 | 0.0 | 62.25 | −1.83 | 2.40 |
| Embodiment 3 | 0.778 | 1.219 | 14.83 | 0.59 | 0.01 | 2 | 0.0 | 65.36 | −0.93 | 0.03 |
| Embodiment 4 | 0.780 | 1.222 | 15.31 | 0.57 | 0.00 | 1 | 0.0 | 65.87 | −2.07 | −0.95 |
| Embodiment 5 | 0.781 | 1.217 | 14.57 | 0.52 | 0.01 | 2 | 0.0 | 65.06 | −1.50 | −1.23 |
| Embodiment 6 | 0.779 | 1.215 | 14.32 | 0.65 | 0.01 | 1 | 0.0 | 64.68 | −1.81 | 1.25 |
| Embodiment 7 | 0.783 | 1.232 | 15.55 | 0.58 | 0.01 | 2 | 0.0 | 64.15 | −0.95 | 0.02 |
| Embodiment 8 | 0.781 | 1.218 | 15.18 | 0.56 | 0.01 | 0 | 0.0 | 66.28 | −1.72 | −1.35 |
| Embodiment 9 | 0.780 | 1.221 | 15.23 | 0.63 | 0.01 | 0 | 0.0 | 63.54 | −1.90 | 1.57 |
| Embodiment 10 | 0.783 | 1.223 | 14.89 | 0.61 | 0.00 | 0 | 0.0 | 65.86 | −1.25 | −0.97 |
| Embodiment 11 | 0.778 | 1.215 | 15.03 | 0.57 | 0.01 | 0 | 0.0 | 67.07 | −2.35 | −1.62 |
| Embodiment 12 | 0.781 | 1.226 | 15.27 | 0.59 | 0.01 | 0 | 0.0 | 66.18 | −1.76 | −1.89 |
| Embodiment 13 | 0.779 | 1.224 | 15.19 | 0.62 | 0.01 | 1 | 0.0 | 65.54 | −1.94 | 0.68 |
| Embodiment 14 | 0.780 | 1.228 | 15.87 | 0.64 | 0.01 | 1 | 0.0 | 65.77 | −1.32 | −0.55 |
| Comparative | 0.778 | 1.220 | 15.89 | 0.45 | 0.01 | 1 | 0.0 | 66.83 | −0.98 | −1.89 |
| example 1 | | | | | | | | | | |
| Comparative | 0.779 | 1.195 | 14.62 | 0.58 | 0.02 | 1 | 0.0 | 65.36 | −1.57 | −1.28 |
| example 2 | | | | | | | | | | |
| Comparative | 0.780 | 1.221 | 15.35 | 0.62 | 0.00 | 3 | 0.1 | 54.62 | −3.26 | 1.55 |
| example 3 | | | | | | | | | | |
| Comparative | 0.778 | 1.170 | 15.22 | 0.49 | 0.02 | 1 | 0.0 | 58.01 | −0.86 | 1.65 |
| example 4 | | | | | | | | | | |

TABLE 2
Physical and chemical indicators of PCTG

| | | | | | | | Agglomerated | | | |
|---|---|---|---|---|---|---|---|---|---|---|
| | IV/ | DEG/ | End carboxyl | H₂O/ | Ash/ | Fe/ | particles ≤10 | Hue | | |
| | dL/g | % | content/mol/t | % | % | mg/kg | μm/% | L | a | b |
| Embodiment 1 | 0.802 | 0.365 | 15.92 | 0.58 | 0.01 | 1 | 0.0 | 65.64 | −1.89 | 0.10 |
| Embodiment 2 | 0.799 | 0.371 | 15.57 | 0.70 | 0.01 | 2 | 0.0 | 63.25 | −1.82 | 2.44 |
| Embodiment 3 | 0.801 | 0.369 | 14.85 | 0.59 | 0.01 | 1 | 0.0 | 64.36 | −0.97 | 1.03 |
| Embodiment 4 | 0.804 | 0.382 | 15.31 | 0.57 | 0.01 | 1 | 0.0 | 66.08 | −2.07 | −0.95 |
| Embodiment 5 | 0.795 | 0.367 | 14.54 | 0.52 | 0.01 | 1 | 0.0 | 64.82 | −1.89 | 1.23 |
| Embodiment 6 | 0.799 | 0.375 | 14.32 | 0.63 | 0.01 | 2 | 0.0 | 65.29 | −1.86 | 0.25 |
| Embodiment 7 | 0.802 | 0.373 | 15.51 | 0.58 | 0.01 | 1 | 0.0 | 64.77 | −0.95 | 0.02 |
| Embodiment 8 | 0.803 | 0.375 | 14.98 | 0.67 | 0.00 | 1 | 0.0 | 66.36 | −1.97 | −0.75 |
| Embodiment 9 | 0.800 | 0.370 | 15.02 | 0.59 | 0.01 | 1 | 0.0 | 64.22 | −1.92 | 1.03 |
| Embodiment 10 | 0.798 | 0.371 | 14.89 | 0.62 | 0.00 | 1 | 0.0 | 65.76 | −1.35 | 0.45 |
| Embodiment 11 | 0.803 | 0.377 | 15.13 | 0.60 | 0.00 | 1 | 0.0 | 67.02 | −2.44 | −1.29 |
| Embodiment 12 | 0.799 | 0.372 | 14.96 | 0.62 | 0.00 | 1 | 0.0 | 65.85 | −1.96 | −1.75 |
| Embodiment 13 | 0.796 | 0.370 | 15.02 | 0.55 | 0.01 | 1 | 0.0 | 65.01 | −2.02 | −0.65 |
| Embodiment 14 | 0.801 | 0.366 | 15.41 | 0.63 | 0.00 | 1 | 0.0 | 65.32 | −1.56 | −0.77 |
| Comparative | 0.802 | 0.386 | 15.89 | 0.55 | 0.01 | 1 | 0.0 | 66.80 | −1.98 | −1.82 |
| example 1 | | | | | | | | | | |
| Comparative | 0.803 | 0.379 | 14.65 | 0.58 | 0.02 | 1 | 0.0 | 65.75 | −1.57 | −1.55 |
| example 2 | | | | | | | | | | |
| Comparative | 0.806 | 0.382 | 15.37 | 0.64 | 0.01 | 2 | 0.1 | 55.47 | −0.86 | 1.55 |
| example 3 | | | | | | | | | | |
| Comparative | 0.800 | 0.378 | 14.29 | 0.59 | 0.02 | 1 | 0.2 | 57.66 | −0.16 | 1.65 |
| example 4 | | | | | | | | | | |

It can be seen that when using the specific soluble germanium chelate catalyst of the present disclosure for catalyzing the polymerization of PETG or PCTG, the resulting copolyester product has high viscosity and excellent hue, which is superior to traditional germanium dioxide catalysts, titanium-based catalysts, or titanium-antimony composite catalysts, or, when citric acid is used, due to its three carboxyl groups, in addition to the two carboxyl groups used for germanium chelation, the germanium chelate also contains a free carboxyl group, which slightly affects the hue of the copolyester product, making its hue comparable to when using traditional catalysts, however, the solubility and stability of the catalyst are significantly improved, without affecting the long-term operation of the polymerization device. And the catalyst of the present disclosure has higher catalytic activity and better catalytic efficiency, and when achieving the same catalytic efficiency, the amount is reduced compared to traditional germanium-based catalysts, thereby reducing the catalyst cost. By using recrystallization to remove acid, the purity of the catalyst obtained is higher and the catalytic efficiency is higher compared to adding a Lewis base to remove acid.

The embodiments described above are only for illustrating the technical concepts and features of the present disclosure, and are intended to make a person familiar with the technology being able to understand the content of the present disclosure and thereby implement it, and should not limit the protective scope of this disclosure. Any equivalent variations or modifications according to the spirit of the present disclosure should be covered by the protective scope of the present disclosure.

What is claimed is:

1. A method for preparing a soluble germanium chelate catalyst, wherein, the preparation method comprises steps of: reacting germanium dioxide with an aqueous solution of an acid to generate a clear solution containing a germanium chelate, wherein the clear solution comprises excess acid; adding a Lewis base to the clear solution to neutralize the excess acid to obtain an aqueous solution of the germanium chelate; post-treating the aqueous solution of the germanium chelate to give the soluble germanium chelate catalyst; the acid is selected from the group consisting of diacids, and C3-C6 diacids or triacids containing hydroxyl or amino group, and combinations thereof.

2. The method for preparing a soluble germanium chelate catalyst according to claim 1, wherein, the acid is selected from the group consisting of citric acid, tartaric acid, glutamic acid, gluconic acid, L-lactic acid, oxalic acid, and combinations thereof.

3. The method for preparing a soluble germanium chelate catalyst according to claim 1, wherein, the structure of germanium dioxide is selected from hexagonal crystal form, anatase form or amorphous form; and/or, the particle size of germanium dioxide ranges from 0.1˜75 μm.

4. The method for preparing a soluble germanium chelate catalyst according to claim 1, wherein, the molar ratio of germanium dioxide to the acid is 1:(1.0˜5.0).

5. The method for preparing a soluble germanium chelate catalyst according to claim 1, wherein, the reaction is carried out at 30˜100° C.; and/or, the reaction is carried out for 2˜6 hours; and/or, the reaction is carried out in a reactor provided with a reflow cooling system, and the water vapor generated by the reaction is refluxed to the reactor; as the reaction proceeds, germanium dioxide gradually reacts to form the germanium chelate and dissolves in water, and the reaction system gradually changes from a white suspension to a clear and transparent state.

6. The method for preparing a soluble germanium chelate catalyst according to claim 1, wherein, in the aqueous solution of the germanium chelate, the mass concentration of the germanium chelate is 0.5˜12.0%; the post-treatment comprises a step of adding ethylene glycol to the aqueous solution of the germanium chelate to obtain a germanium chelate solution in a mixed solution of water and ethylene glycol.

7. The method for preparing a soluble germanium chelate catalyst according to claim 1, wherein, after neutralization with a Lewis base, the pH value of the clear solution is 5.5˜7.0.

8. The method for preparing a soluble germanium chelate catalyst according to claim 1, wherein, the Lewis base is selected from the group consisting of sodium hydroxide, lithium hydroxide, potassium hydroxide, sodium acetate, calcium acetate, zinc acetate, lithium acetate, manganese acetate, cobalt acetate, sodium carbonate, sodium bicarbonate, magnesium hydroxide, aluminum hydroxide, and combinations thereof.

9. The method for preparing a soluble germanium chelate catalyst according to claim 1, wherein, the post-treatment comprises steps of cooling, standing for crystallization, filtering, washing, and drying the aqueous solution of the germanium chelate to obtain a germanium chelate crystal.

10. The method for preparing a soluble germanium chelate catalyst according to claim 9, wherein, the temperature after cooling is −20˜0° C.; and/or, the washing is carried out in anhydrous alcohol at 50˜70° C., and excess acid is further removed through washing.

11. The method for preparing a soluble germanium chelate catalyst according to claim 9, wherein, the post-treatment further comprises steps of dissolving the germanium chelate crystal in a certain amount of water under agitating conditions at 60˜80° C., adding ethylene glycol after complete dissolution, and continuing to agitate evenly to obtain the catalyst; and/or, in the soluble germanium chelate catalyst, the mass ratio of water to ethylene glycol is 1:4˜4:1.

12. The method for preparing a soluble germanium chelate catalyst according to claim 11, wherein, in the catalyst, the mass concentration of the germanium chelate is 0.5%˜14.0%.

13. A method for synthesizing a PETG or PCTG polyester, wherein, the preparation method comprises steps of: reacting germanium dioxide with an aqueous solution of an acid to generate a clear solution containing a germanium chelate, wherein the clear solution comprises excess acid; adding a Lewis base to the clear solution to neutralize the excess acid to obtain an aqueous solution of the germanium chelate; post-treating the aqueous solution of the germanium chelate to give a soluble germanium chelate catalyst; the acid is selected from the group consisting of diacids, and C3-C6 diacids or triacids containing hydroxyl or amino group, and combinations thereof; using terephthalic acid, ethylene glycol, and 1,4-cyclohexanedimethanol as polymerization monomers, conducting esterification and polymerization reactions in the presence of a catalyst to obtain the PETG or PCTG, where the catalyst comprises the soluble germanium chelate catalyst.

14. The method for synthesizing a PETG or PCTG polyester according to claim 13, wherein, the catalyst is the soluble germanium chelate catalyst.

15. The method for synthesizing a PETG or PCTG polyester according to claim 14, wherein, in terms of germanium dioxide, the soluble germanium chelate catalyst used to catalyze the esterification reaction accounts for 5˜15 ppm of the mass of PETG or PCTG, and the soluble germanium chelate catalyst used to catalyze the polymerization reaction accounts for 50˜70 ppm of the mass of PETG or PCTG.

16. The method for synthesizing a PETG or PCTG polyester according to claim 13, wherein, the polymerization catalyst comprises the soluble germanium chelate catalyst and a titanium-based catalyst.

17. The method for synthesizing a PETG or PCTG polyester according to claim 16, wherein, the titanium element in the titanium-based catalyst and the germanium element in the soluble germanium chelate catalyst account for 1˜35 ppm and 150˜10 ppm of the mass of PETG or PCTG, respectively.
